(19) United States
(12) Patent Application Publication
(10) Pub. No.: US 2025/0003067 A1
(43) Pub. Date: Jan. 2, 2025

(54) FILM QUALITY IMPROVER, METHOD OF FORMING THIN FILM USING FILM QUALITY IMPROVER, AND SEMICONDUCTOR SUBSTRATE FABRICATED USING METHOD

(71) Applicant: SOULBRAIN CO., LTD., Gyeonggi-do (KR)

(72) Inventors: Jae Sun JUNG, Gyeonggi-do (KR); Chang Bong YEON, Gyeonggi-do (KR); Seung Hyun LEE, Gyeonggi-do (KR); Jong Moon KIM, Gyeonggi-do (KR)

(21) Appl. No.: 18/691,435

(22) PCT Filed: Sep. 13, 2022
(86) PCT No.: PCT/KR2022/013587

§ 371 Date: Mar. 12, 2024

(30) Foreign Application Priority Data
Sep. 13, 2021 (KR) 10-2021-0121705
Oct. 29, 2021 (KR) 10-2021-0146536

(51) Int. Cl. C23C 16/455; C07C 19/07; C07C 19/08; C07C 19/10; C07C 19/14; C23C 16/34; C23C 16/44; H01L 21/285; H01L 21/768; H01L 23/532

(52) U.S. Cl. CPC C23C 16/45534; C07C 19/07; C07C 19/08; C07C 19/10; C07C 19/14; C23C 16/34; C23C 16/4408; H01L 21/28556; H01L 21/76841; H01L 23/53238

(57) ABSTRACT

The present invention relates to a film quality improver, a method of forming a thin film using the film quality improver, and a semiconductor substrate fabricated using the method. According to the present invention, side reactions may be suppressed by using a film quality improver having a predetermined structure in a thin film deposition process, and process by-products in a thin film may be removed by appropriately controlling a thin film growth rate. As a result, even when the thin film is formed on a substrate having a complicated structure, step coverage and the thickness uniformity of the thin film may be greatly improved. In addition, corrosion or deterioration may be prevented, and the electrical properties of the thin film may be improved due to improvement in the crystallinity of the thin film.

### TECHNICAL FIELD

The present invention relates to a film quality improver, a method of forming a thin film using the film quality improver, and a semiconductor substrate fabricated using the method. More particularly, the present invention relates to a film quality improver capable of inhibiting side reactions to reduce the concentration of impurities in a thin film, preventing corrosion or deterioration of a thin film to improve the electrical properties of the thin film, and controlling the growth rate of a thin film to greatly improve step coverage and the thickness uniformity of the thin film even when the thin film is formed on a substrate having a complicated structure, a method of forming a thin film using the film quality improver, and a semiconductor substrate fabricated using the method.

### BACKGROUND ART

Development of high-integration memory and non-memory semiconductor devices is actively progressing. As the structures of memory and non-memory semiconductor devices become increasingly complex, the importance of step coverage is gradually increasing in depositing various thin films on substrates.

The thin film for semiconductors is made of a metal nitride, a metal oxide, a metal silicide, or the like. Examples of the metal nitride include titanium nitride (TiN), tantalum nitride (TaN), zirconium nitride (ZrN), and the like. The thin film is generally used as a diffusion barrier between a silicon layer of a doped semiconductor and aluminum (Al) or copper (Cu) used as an interlayer wiring material. However, when depositing a tungsten (W) thin film on a substrate, the thin film serves as an adhesive layer.

To impart excellent and uniform physical properties to a thin film deposited on a substrate, the formed thin film must have high step coverage. Accordingly, the atomic layer deposition (ALD) process using surface reaction is used rather than the chemical vapor deposition (CVD) process using gas phase reaction, but there are still problems to be solved to realize the desired step coverage.

In addition, a method of reducing the growth rate of a thin film has been proposed as a method to improve step coverage. When a deposition temperature is reduced to decrease the growth rate of a thin film, the residual amount of impurities such as carbon or chlorine in the thin film increases, causing a significant decrease in film quality.

In addition, in the case of titanium tetrachloride (TiCl₄) used to deposit titanium nitride (TiN), which is a typical metal nitride, process by-products such as chlorides remain in a formed thin film, causing corrosion of metals such as aluminum. In addition, film quality is reduced due to generation of non-volatile by-products.

Therefore, it is necessary to develop a method of forming a thin film having a complex structure that has low residual by-products and does not cause corrosion of interlayer wiring materials and a semiconductor substrate fabricated by the method.

### RELATED ART DOCUMENTS

### Patent Documents

KR 2006-0037241 A

### DISCLOSURE

### Technical Problem

Therefore, the present invention has been made in view of the above problems, and it is one object of the present invention to provide a thin film-forming growth inhibitor capable of inhibiting side reactions to appropriately reduce the growth rate of a thin film and capable of removing process by-products from a thin film to prevent corrosion or deterioration and greatly improve step coverage and the thickness uniformity of the thin film even when the thin film is formed on a substrate having a complicated structure, a method of forming a thin film using the growth inhibitor, and a semiconductor substrate fabricated by the method.

It is another object of the present invention to improve the density and electrical properties of a thin film by improving the crystallinity of the thin film.

The above and other objects can be accomplished by the present invention described below.

### Technical Solution

In accordance with one aspect of the present invention, provided is a film quality improver, wherein the film quality improver is a compound represented by Chemical Formula 1 below:

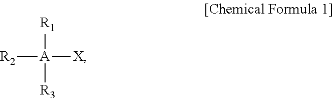

wherein A is carbon or silicon, R₁, R₂, and R₃ are independently alkyl groups having 1 to 3 carbon atoms, one or more of R₁, R₂, and R₃ has 2 or 3 carbon atoms, and X includes one or more of fluorine (F), chlorine (Cl), bromine (Br), and iodine (I).

In accordance with another aspect of the present invention, provided is a method of forming a thin film, the method including injecting the film quality improver into an ALD chamber and adsorbing the film quality improver on a surface of a substrate loaded into the ALD chamber.

In accordance with yet another aspect of the present invention, provided is a semiconductor substrate fabricated using the method of forming a thin film.

### Advantageous Effects

According to the present invention, the present invention has an effect of providing a film quality improver for forming a thin film with a predetermined structure that is capable of improving step coverage and the density of a thin film and reducing process by-products during formation of the thin film. In addition, the present invention has an effect of providing a method of forming a thin film using the film quality improver and a semiconductor substrate fabricated using the method.

In addition, according to the present invention, the present invention can improve the density and electrical properties of a thin film by improving the crystallinity of the thin film.

### DESCRIPTION OF DRAWINGS

FIG. 1 is a graph showing the average growth rate of the film formation processes according to Examples 1 and 2 of the present invention and Comparative Examples 1 to 4.

FIG. 2 is a graph showing the electrical resistance characteristics (resistivity) of the thin films formed according to Examples 1 and 2 of the present invention and Comparative Examples 1 to 4.

### BEST MODE

Hereinafter, a film quality improver according to the present invention, a method of forming a thin film using the film quality improver, and a semiconductor substrate fabricated using the method will be described in detail.

The present inventors confirmed that, when a halogen-substituted branched compound having a predetermined structure was adsorbed before adsorbing a thin film precursor compound on the surface of a substrate loaded into an ALD chamber, the growth rate of a thin film formed after deposition was greatly reduced, and thus step coverage was greatly improved, and halides remaining as process by-products were greatly reduced. In addition, the present inventors confirmed that, when a thin film precursor compound was adsorbed onto the surface of a substrate loaded into an ALD chamber, and then a halogen-substituted branched compound having a predetermined structure was adsorbed, contrary to the previous case, the growth rate of a thin film formed after deposition was increased, halides remaining as process by-products were greatly reduced, and the density and resistivity of the thin film were greatly improved. Based on these results, the present inventors conducted further studies to complete the present invention.

The film quality improver of the present invention is a compound represented by Chemical Formula 1 below.

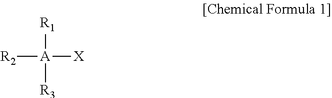

In Chemical Formula 1, A is carbon or silicon, R₁, R₂, and R₃ are independently alkyl groups having 1 to 3 carbon atoms, one or more of R₁, R₂, and R₃ has 2 or 3 carbon atoms, and X includes one or more of fluorine (F), chlorine (Cl), bromine (Br), and iodine (I). In this case, side reactions may be suppressed during formation of a thin film, and the growth rate of the thin film may be controlled, thereby preventing corrosion or deterioration due to process by-products in the thin film and improving the crystallinity of the thin film. In addition, even when a thin film is formed on a substrate having a complicated structure, step coverage and the thickness uniformity of the thin film may be greatly improved.

In Chemical Formula 1, A is carbon or silicon, preferably carbon.

R₁, R₂, and R₃ are each independently alkyl groups having 1 to 3 carbon atoms, and one or more of R₁, R₂, and R₃ has 2 or 3 carbon atoms. As a preferred example, one or more of R₁, R₂, and R₃ has 1 carbon atom and the remaining two have 2 or 3 carbon atoms. More preferably, any one of R₁, R₂, and R₃ has 1 carbon atom and the remaining two has 2 carbon atoms. Within this range, process by-products may be greatly reduced, step coverage may be excellent, and the density and electrical properties of a thin film may be improved.

In Chemical Formula 1, X may be a halogen element, preferably fluorine, chlorine, or bromine, more preferably chlorine or bromine. Within this range, process by-products may be reduced and step coverage may be improved. In addition, X may be fluorine. This case may be more suitable for processes requiring high temperature deposition.

In Chemical Formula 1, as a preferred example, X may be iodine. Within this range, thin film crystallinity may be improved, and side reactions may be suppressed, thereby reducing process by-products.

The compound represented by Chemical Formula 1 may be a halogen-substituted tertiary alkyl compound. As a specific example, the compound may include one or more selected from the group consisting of 2-chloro-2-methylbutane, 2-chloro-2-methylpentane, 3-chloro-3-methylpentane, 3-chloro-3-methylhexane, 3-chloro-3-ethylpentane, 3-chloro-3-ethylhexane, 4-chloro-4-methylheptane, 4-chloro-4-ethylheptane, 4-chloro-4-propylheptane, 2-bromo-2-methylbutane, 2-bromo-2-methylpentane, 3-bromo-3-methylpentane, 3-bromo-3-methylhexane, 3-bromo-3-ethylpentane, 3-bromo-3-ethylhexane, 4-bromo-4-methylheptane, 4-bromo-4-ethylheptane, 4-bromo-4-propylheptane, 2-iodo-2-methylbutane, 2-iodo-2-methylpentane, 3-iodo-3-methylpentane, 3-iodo-3-methylhexane, 3-iodo-3-ethylpentane, 3-iodo-3-ethylhexane, 4-iodo-4-methylheptane, 4-iodo-4-ethylheptane, 4-iodo-4-propylheptane, 2-fluoro-2-methylbutane, 2-fluoro-2-methylpentane, 3-fluoro-3-methylpentane, 3-fluoro-3-methylhexane, 3-fluoro-3-ethylpentane, 3-fluoro-3-ethylhexane, 4-fluoro-4-methylheptane, 4-fluoro-4-ethylheptane, and 4-fluoro-4-propylheptane, preferably one or more selected from the group consisting of 2-chloro-2-methylbutane and 3-chloro-3-methylpentane. In this case, the effect of removing process by-products may be increased, and step coverage and film quality may be improved.

The compound represented by Chemical Formula 1 is preferably used in an atomic layer deposition (ALD) process. In this case, the compound may effectively protect the surface of a substrate by acting as a film quality improver without interfering with adsorption of the thin film precursor compound, and process by-products may be effectively removed.

Preferably, the compound represented by Chemical Formula 1 may be in a liquid state at room temperature (22° C.) and may have a density of 0.8 to 2.5 g/cm³ or 0.8 to 1.5 g/cm³, a vapor pressure (20° C.) of 0.1 to 300 mmHg or 1 to 300 mmHg, and a solubility (25° C.) of 200 mg/L or less in water. Within this range, step coverage, the thickness uniformity of a thin film, and film quality may be excellent.

More preferably, the compound represented by Chemical Formula 1 may have a density of 0.75 to 2.0 g/cm³ or 0.8 to 1.3 g/cm³, a vapor pressure (20° C.) of 1 to 260 mmHg, and a solubility (25° C.) of 160 mg/L or less in water. Within this range, step coverage, the thickness uniformity of a thin film, and film quality may be improved.

The method of forming a thin film of the present invention includes a step of injecting a film quality improver represented by Chemical Formula 1 below into an ALD chamber and adsorbing the film quality improver on the surface of a substrate loaded into the ALD chamber.

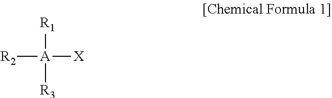

In Chemical Formula 1, A is carbon or silicon, R₁, R₂, and R₃ are independently alkyl groups having 1 to 3 carbon atoms, one or more of R₁, R₂, and R₃ has 2 or 3 carbon atoms, and X includes one or more of fluorine (F), chlorine (Cl), bromine (Br), and iodine (I). In this case, side reactions may be suppressed during formation of a thin film, and the growth rate of the thin film may be controlled, thereby reducing process by-products in the thin film, preventing corrosion or deterioration, and improving the crystallinity of the thin film. Thus, even when the thin film is formed on a substrate having a complicated structure, step coverage and the electrical properties of the thin film may be greatly improved.

In the step of adsorbing the film quality improver on the surface of a substrate, when the film quality improver is fed onto the surface of the substrate, the feeding time per cycle may be preferably 0.01 to 5 seconds, more preferably 0.02 to 3 seconds, still more preferably 0.04 to 2 seconds, still more preferably 0.05 to 1 second. Within this range, the growth rate of a thin film may be reduced, and step coverage and economics may be excellent.

In the present disclosure, the feeding time of the film quality improver is determined based on a chamber volume of 15 to 20 L and a flow rate of 0.5 to 5 mg/s, more specifically, based on a chamber volume of 18 L and a flow rate of 1 to 2 mg/s.

As a preferred example, the method of forming a thin film may include step i) of vaporizing the film quality improver and adsorbing the film quality improver on the surface of a substrate loaded into an ALD chamber; step ii) of performing first purging of the inside of the ALD chamber using a purge gas; step iii) of vaporizing a thin film precursor compound and adsorbing the precursor compound on the surface of the substrate loaded into the ALD chamber; step iv) of performing second purging of the inside of the ALD chamber using a purge gas; step v) of supplying a reaction gas into the ALD chamber; and step vi) of performing third purging of the inside of the ALD chamber using a purge gas. At this time, when setting step i) to step vi) as a unit cycle, the unit cycle may be repeated until a thin film of a desired thickness is obtained. In this way, within one cycle, when the film quality improver of the present invention is added before the thin film precursor compound and adsorbed to the substrate, even when deposited at high temperature, the growth rate of a thin film may be appropriately reduced. In addition, generated process by-products may be effectively eliminated, thereby reducing the resistivity of the thin film and greatly improving step coverage.

As another example, the method of forming a thin film may include step i) of vaporizing a thin film precursor compound and adsorbing the thin film precursor compound on the surface of a substrate loaded into an ALD chamber; step ii) of performing first purging of the inside of the ALD chamber using a purge gas; step iii) of vaporizing the film quality improver and adsorbing the film quality improver on the surface of the substrate loaded into the ALD chamber; step iv) of performing second purging of the inside of the ALD chamber using a purge gas; step v) of supplying a reaction gas into the ALD chamber; and step vi) of performing third purging of the inside of the ALD chamber using a purge gas. At this time, when setting step i) to step vi) as a unit cycle, the unit cycle may be repeated until a thin film of a desired thickness is obtained. In this way, within one cycle, when the film quality improver of the present invention is added after the thin film precursor compound and adsorbed to the substrate, the growth rate of a thin film may be increased. In addition, the density and crystallinity of the thin film may be increased, thereby reducing the resistivity of the thin film and greatly improving electrical properties.

As a preferred example, according to the method of forming a thin film of the present invention, within one cycle, the film quality improver of the present invention may be supplied before the thin film precursor compound and adsorbed to the substrate. In this case, even when the thin film is deposited at high temperatures, the growth rate of the thin film may be appropriately reduced, thereby greatly reducing process by-products and greatly improving step coverage. In addition, the crystallinity of the thin film may be increased, thereby reducing the resistivity of the thin film. In addition, even when the thin film is applied to a semiconductor device with a large aspect ratio, since the thickness uniformity of the thin film is greatly improved, the reliability of the semiconductor device may be secured.

For example, according to the method of forming a thin film, when depositing the film quality improver before or after deposition of the thin film precursor compound, when necessary, the unit cycle may be repeated 1 to 99,999 times, preferably 10 to 10,000 times, more preferably 50 to 5,000 times, still more preferably 100 to 2,000 times. Within this range, a thin film having a desired thickness may be obtained, and the effects desired to be achieved in the present invention may be fully achieved.

When the film quality improver is first adsorbed onto the substrate and then the thin film precursor compound is adsorbed, or when the thin film precursor compound is first adsorbed and then the film quality improver is adsorbed, in the step of purging the non-adsorbed film quality improver, an amount of the purge gas introduced into the ALD chamber is not particularly limited as long as the amount is sufficient to remove the non-adsorbed film quality improver. For example, the amount of the purge gas may be 10 to 100,000 times, preferably 50 to 50,000 times, more preferably 100 to 10,000 times. Within this range, the non-adsorbed film quality improver is sufficiently removed so that a thin film may be formed evenly and deterioration of film quality may be prevented. Here, the input amounts of the purge gas and film quality improver are each based on one cycle, and the volume of the film quality improver refers to the volume of the vaporized film quality improver.

As a specific example, the film quality improver may be injected (per cycle) at a flow rate of 1.66 mL/s and an injection time of 0.5 sec. In the step of purging the non-adsorbed film quality improver, when the purge gas is injected (per cycle) at a flow rate of 166.6 mL/s and an injection time of 3 sec, the injection amount of the purge gas is 602 times that of the film quality improver.

In addition, in the step of purging the non-adsorbed thin film precursor compound, an amount of the purge gas introduced into the ALD chamber is not particularly limited as long as the amount is sufficient to remove the non-adsorbed thin film precursor compound. For example, based on the volume of the thin film precursor compound introduced into the ALD chamber, the amount of the purge gas may be 10 to 10,000 times, preferably 50 to 50,000 times, more preferably 100 to 10,000 times. Within this range, the non-adsorbed thin film precursor compound is sufficiently removed so that a thin film may be formed evenly and deterioration of film quality may be prevented. Here, the input amounts of the purge gas and thin film precursor compound are each based on one cycle, and the volume of the thin film precursor compound refers to the volume of the vaporized thin film precursor compound.

In addition, in the purging step performed immediately after the reaction gas supply step, based on the volume of the reaction gas introduced into the ALD chamber, the amount of the purge gas introduced into the ALD chamber may be 10 to 10,000 times, preferably 50 to 50,000 times, more preferably 100 to 10,000 times. Within this range, the desired effect may be sufficiently obtained. Here, the input amounts of the purge gas and reaction gas are each based on one cycle.

The film quality improver and the thin film precursor compound may preferably be transferred into an ALD chamber by a VFC method, a DLI method, or an LDS method, more preferably an LDS method.

When the film quality improver and the precursor compound are fed into the ALD chamber, the input amount ratio (mg/cycle) of the film quality improver to the precursor compound may be preferably 1:1.5 to 1:20, more preferably 1:2 to 1:15, still more preferably 1:2 to 1:12, still more preferably 1:2.5 to 1:10. Within this range, step coverage may be improved, and process by-products may be reduced.

Thin film precursor compounds commonly used in an atomic layer deposition (ALD) method may be used as the thin film precursor compound according to the present invention without particular limitation. Preferably, the thin film precursor compound may include one or more selected from the group consisting of a metal film precursor compound, a metal oxide film precursor compound, a metal nitride film precursor compound, and a silicon nitride film precursor compound. The metal may include preferably one or more selected from the group consisting of tungsten, cobalt, chromium, aluminum, hafnium, vanadium, niobium, germanium, lanthanides, actinoids, gallium, tantalum, zirconium, ruthenium, copper, titanium, nickel, iridium, and molybdenum.

For example, the metal film precursor, the metal oxide film precursor, and the metal nitride film precursor may independently include one or more selected from the group consisting of metal halides, metal alkoxides, alkyl metal compounds, metal amino compounds, metal carbonyl compounds, and substituted or unsubstituted cyclopentadienyl metal compounds, without being limited thereto.

As a specific example, the metal film precursor, the metal oxide film precursor, and the metal nitride film precursor may independently include one or more selected from the group consisting of tetrachlorotitanium, tetrachlorogemanium, tetrchlorotin, tris(isopropyl)ethylmethyl aminogermanium, tetraethoxylgermanium, tetramethyl tin, tetraethyl tin, bisacetylacetonate tin, trimethylaluminum, tetrakis(dimethylamino) germanium, bis(n-butylamino) germanium, tetrakis(ethylmethylamino) tin, tetrakis(dimethylamino) tin, dicobalt octacarbonyl (Co₂(CO)₈), biscyclopentadienylcobalt (Cp₂Co), cobalt tricarbonyl nitrosyl (Co(CO)₃(NO)), and cabalt dicarbonyl cyclopentadienyl (CpCo(CO)₂), without being limited thereto.

For example, the silicon nitride film precursor may include one or more selected from the group consisting of SiH₄, SiCl₄, SiF₄, SiCl₂H₂, Si₂Cl₆, TEOS, DIPAS, BTBAS, (NH₂)Si(NHMe)₃, (NH₂)Si(NHEt)₃, (NH₂)Si(NHⁿPr)₃, (NH₂)Si(NHⁱPr)₃, (NH₂)Si(NHⁿBu)₃, (NH₂)Si(NHⁱBu)₃, (NH₂)Si(NH^{t}Bu)₃, (NMe₂)Si(NHMe)₃, (NMe₂)Si(NHEt)₃, (NMe₂)Si(NHⁿPr)₃, (NMe₂)Si(NHⁱPr)₃, (NMe₂)Si(NHⁿBu)₃, (NMe₂)Si(NHⁱBu)₃, (NMe₂)Si(NH^{t}Bu)₃, (NEt₂)Si(NHMe)₃, (NEt₂)Si(NHEt)₃, (NEt₂)Si(NHⁿPr)₃, (NEt₂)Si(NHⁱPr)₃, (NEt₂)Si(NHⁿBu)₃, (NEt₂)Si(NHⁱBu)₃, (NEt₂)Si(NH^{t}Bu)₃, (NⁿPr₂)Si(NHMe)₃, (NⁿPr₂)Si(NHEt)₃, (NⁿPr₂)Si(NHⁿPr)₃, (NⁿPr₂)Si(NHⁱPr)₃, (NⁿPr₂)Si(NHⁿBu)₃, (NⁿPr₂)Si(NHⁱBu)₃, (NⁿPr₂)Si(NH^{t}Bu)₃, (NⁱPr₂)Si(NHMe)₃, (NⁱPr₂)Si(NHEt)₃, (NⁱPr₂)Si(NHⁿPr)₃, (NⁱPr₂)Si(NHⁱPr)₃, (NⁱPr₂)Si(NHⁿBu)₃, (NⁱPr₂)Si(NHⁱBu)₃, (NⁱPr₂)Si(NH^{t}Bu)₃, (NⁿBu₂)Si(NHMe)₃, (NⁿBu₂)Si(NHEt)₃, (NⁿBu₂)Si(NHⁿPr)₃, (NⁿBu₂)Si(NHⁱPr)₃, (NⁿBu₂)Si(NHⁿBu)₃, (NⁿBu₂)Si(NHⁱBu)₃, (NⁿBu₂)Si(NH^{t}Bu)₃, (NⁱBu₂)Si(NHMe)₃, (NⁱBu₂)Si(NHEt)₃, (NⁱBu₂)Si(NHⁿPr)₃, (NⁱBu₂)Si(NHⁱPr)₃, (NⁱBu₂)Si(NHⁿBu)₃, (NⁱBu₂)Si(NHⁱBu)₃, (NⁱBu₂)Si(NH Bu)₃, (N^{t}Bu₂)Si(NHMe)₃, (N^{t}Bu₂)Si(NHEt)₃, (N^{t}Bu₂)Si(NHⁿPr)₃, (N^{t}Bu₂)Si(NH Pr)₃, (N^{t}Bu₂)Si(NHⁿBu)₃, (N^{t}Bu₂)Si(NHⁱBu)₃, (N^{t}Bu₂)Si(NH Bu)₃, (NH₂)₂Si(NHMe)₂, (NH₂)₂Si(NHEt)₂, (NH₂)₂Si(NHⁿPr)₂, (NH₂)₂Si(NHⁱPr)₂, (NH₂)₂Si(NHⁿBu)₂, (NH₂)₂Si(NH Bu)₂, (NH₂)₂Si(NH^{t}Bu)₂, (NMe₂)₂Si(NHMe)₂, (NMe₂)₂Si(NHEt)₂, (NMe₂)₂Si(NHⁿPr)₂, (NMe₂)₂Si(NHⁱPr)₂, (NMe₂)₂Si(NHⁿBu)₂, (NMe₂)₂Si(NHⁱBu)₂, (NMe₂)₂Si(NH Bu)₂, (NEt₂)₂Si(NHMe)₂, (NEt₂)₂Si(NHEt)₂, (NEt₂)₂Si(NHⁿPr)₂, (NEt₂)₂Si(NH Pr)₂, (NEt₂)₂Si(NHⁿBu)₂, (NEt₂)₂Si(NHⁱBu)₂, (NEt₂)₂Si(NH Bu)₂, (NⁿPr₂)₂Si(NHMe)₂, (NⁿPr₂)₂Si(NHEt)₂, (NⁿPr₂)₂Si(NHⁿPr)₂, (NⁿPr₂)₂Si(NHⁱPr)₂, (NⁿPr₂)₂Si(NHⁿBu)₂, (NⁿPr₂)₂Si(NHⁱBu)₂, (NⁿPr₂)₂Si(NH^{t}Bu)₂, (NⁱPr₂)₂Si(NHMe)₂, (NⁱPr₂)₂Si(NHEt)₂, (NⁱPr₂)₂Si(NHⁿPr)₂, (NⁱPr₂)₂Si(NHⁱPr)₂, (NⁱPr₂)₂Si(NHⁿBu)₂, (NⁱPr₂)₂Si(NH Bu)₂, (NⁱPr₂)₂Si(NH Bu)₂, (NⁿBu₂)₂Si(NHMe)₂, (NⁿBu₂)₂Si(NHEt)₂, (NⁿBu₂)₂Si(NHⁿPr)₂, (NⁿBu₂)₂Si(NHⁱPr)₂, (NⁿBu₂)₂Si(NHⁿBu)₂, (NⁿBu₂)₂Si(NHⁱBu)₂, (NⁿBu₂)₂Si(NH^{t}Bu)₂, (NⁱBu₂)₂Si(NHMe)₂, (NⁱBu₂)₂Si(NHEt)₂, (NⁱBu₂)₂Si(NHⁿPr)₂, (NⁱBu₂)₂Si(NHⁱPr)₂, (NⁱBu₂)₂Si(NHⁿBu)₂, (NⁱBu₂)₂Si(NHⁱBu)₂, (NⁱBu₂)₂Si(NH^{t}Bu)₂, (N^{t}Bu₂)₂Si(NHMe)₂, (N^{t}Bu₂)₂Si(NHEt)₂, (N^{t}Bu₂)₂Si(NHⁿPr)₂, (N^{t}Bu₂)₂Si(NH Pr)₂, (N^{t}Bu₂)₂Si(NHⁿBu)₂, (N^{t}Bu₂)₂Si(NH Bu)₂, (N^{t}Bu₂)₂Si(NH^{t}Bu)₂, Si(HNCH₂CH₂NH)₂, Si(MeNCH₂CH₂NMe)₂, Si(EtNCH₂CH₂NEt)₂, Si(ⁿPrNCH₂CH₂NⁿPr)₂, Si(ⁱPrNCH₂CH₂NⁱPr)₂, Si(ⁿBuNCH₂CH₂NⁿBu)₂, Si(ⁱBuNCH₂CH₂NⁱBu)₂, Si(^{t}BuNCH₂CH₂N^{t}Bu)₂, Si(HNCHCHNH)₂, Si(MeNCHCHNMe)₂, Si(EtNCHCHNEt)₂, Si(ⁿPrNCHCHNⁿPr)₂, Si(ⁱPrNCHCHNⁱPr)₂, Si(ₙBuNCHCHNⁿBu)₂, Si(ⁱBuNCHCHNⁱBu)₂, Si(^{t}BuNCHCHN^{t}Bu)₂, (HNCHCHNH)Si(HNCH₂CH₂NH), (MeNCHCHNMe)Si(MeNCH₂CH₂NMe), (EtNCHCHNEt)Si(EtNCH₂CH₂NEt), (ⁿPrNCHCHNⁿPr)Si(ⁿPrNCH₂CH₂NⁿPr), (ⁱPrNCHCHNⁱPr)Si(ⁱPrNCH₂CH₂NⁱPr), (ⁿBuNCHCHNⁿBu)Si(ⁿBuNCH₂CH₂NⁿBu), (ⁱBuNCHCHNⁱBu)Si(ⁱBuNCH₂CH₂NⁱBu), (^{t}BuNCHCHN^{t}Bu)Si(^{t}BuNCH₂CH₂N^{t}Bu), (NH^{t}Bu)₂Si(HNCH₂CH₂NH), (NH^{t}Bu)₂Si(MeNCH₂CH₂NMe), (NH^{t}Bu)₂Si(EtNCH₂CH₂NEt), (NH^{t}Bu)₂Si(ⁿPrNCH₂CH₂NⁿPr), (NH^{t}Bu)₂Si(ⁱPrNCH₂CH₂NⁱPr), (NH^{t}Bu)₂Si(ⁿBuNCH₂CH₂NⁿBu), (NH^{t}Bu)₂Si(ⁱBuNCH₂CH₂NⁱBu), (NH^{t}Bu)₂Si(BuNCH₂CH₂N^{t}Bu), (NH^{t}Bu)₂Si(HNCHCHNH), (NH^{t}Bu)₂Si(MeNCHCHNMe), (NH^{t}Bu)₂Si(EtNCHCHNEt), (NH^{t}Bu)₂Si(ⁿPrNCHCHNⁿPr), (NH^{t}Bu)₂Si(ⁱPrNCHCHNⁱPr), (NH^{t}Bu)₂Si(^{t}BuNCHCHN^{t}Bu), (NH^{t}Bu)₂Si(ⁱBuNCHCHNⁱBu), (NH^{t}Bu)₂Si(^{t}BuNCHCHN^{t}Bu), (ⁱPrNCH₂CH₂NⁱPr)Si(NHMe)₂, (ⁱPrNCH₂CH₂NⁱPr)Si(NHEt)₂, (ⁱPrNCH₂CH₂NⁱPr)Si(NHⁿPr)₂, (ⁱPrNCH₂CH₂NⁱPr)Si(NHⁱPr)₂, (ⁱPrNCH₂CH₂NⁱPr)Si(NHⁿBu)₂, (ⁱPrNCH₂CH₂NⁱPr)Si(NHⁱBu)₂, (ⁱPrNCH₂CH₂NⁱPr)Si(NH^{t}Bu)₂, (ⁱPrNCHCHNⁱPr)Si(NHMe)₂, (ⁱPrNCHCHNⁱPr)Si(NHEt)₂, (ⁱPrNCHCHNⁱPr)Si(NHⁿPr)₂, (ⁱPrNCHCHNⁱPr)Si(NHⁱPr)₂, (ⁱPrNCHCHNⁱPr)Si(NHⁿBu)₂, (ⁱPrNCHCHNⁱPr)Si(NHⁱBu)₂, and (ⁱPrNCHCHNⁱPr)Si(NH^{t}Bu)₂, without being limited thereto.

Here, ⁿPr means n-propyl, ⁱPr means iso-propyl, ⁿBu means n-butyl, ⁱBu means iso-butyl, and ^{t}Bu means tert-butyl.

As a preferred example, the thin film precursor compound may include one or more selected from the group consisting of TiCl₄, (Ti(CpMe₅)(OMe)₃), Ti(CpMe₃)(OMe)₃, Ti(OMe)₄, Ti(OEt)₄, Ti(OtBu)₄, Ti(CpMe)(OiPr)₃, TTIP(Ti(OiPr)₄, TDMAT(Ti(NMe₂)₄), and Ti(CpMe){N(Me₂)₃}. In this case, the effects desired to be achieved in the present invention may be fully achieved.

The titanium tetrahalide may be used as a metal precursor of a composition for forming a thin film. For example, the titanium tetrahalide may be at least one selected from the group consisting of TiF₄, TiCl₄, TiBr₄, and TiI₄. As a preferred example, considering economic feasibility, the titanium tetrahalide is TiCl₄, but the present invention is not limited thereto.

For example, since the titanium tetrahalide does not decompose at room temperature due to excellent thermal stability thereof and exists in a liquid state, the titanium tetrahalide may be used as a precursor for depositing a thin film according to atomic layer deposition (ALD).

For example, the thin film precursor compound may be fed into a chamber after being mixed with a non-polar solvent. In this case, the viscosity of the thin film precursor compound or vapor pressure may be easily adjusted.

The non-polar solvent may include preferably one or more selected from the group consisting of alkanes and cycloalkanes. In this case, step coverage may be improved even when deposition temperature is increased when forming a thin film while containing an organic solvent having low reactivity and solubility and capable of easy moisture management.

As a more preferred example, the non-polar solvent may include a C1 to C10 alkane or a C3 to C10 cycloalkane, preferably a C3 to C10 cycloalkane. In this case, reactivity and solubility may be reduced, and moisture management may be easy.

In the present disclosure, C1, C3, and the like mean the carbon number.

The cycloalkane may be preferably a C3 to C10 monocycloalkane. Among the monocycloalkanes, cyclopentane exists in a liquid state at room temperature and has the highest vapor pressure, and thus is preferable in a vapor deposition process. However, the present invention is not limited thereto.

For example, the non-polar solvent has a solubility (25° C.) of 200 mg/L or less, preferably 50 to 200 mg/L, more preferably 135 to 175 mg/L in water. Within this range, reactivity to the thin film precursor compound may be low, and moisture management may be easy.

In the present disclosure, solubility may be measured without particular limitation according to measurement methods or standards commonly used in the art to which the present invention pertains. For example, solubility may be measured according to the HPLC method using a saturated solution.

Based on a total weight of the thin film precursor compound and the non-polar solvent, the non-polar solvent may be included in an amount of preferably 5 to 95% by weight, more preferably 10 to 90% by weight, still more preferably 40 to 90% by weight, most preferably 70 to 90% by weight.

When the content of the non-polar solvent exceeds the above range, impurities are generated to increase resistance and impurity levels in a thin film. When the content of the non-polar solvent is less than the above range, an effect of improving step coverage and reducing impurities such as chlorine (Cl) ions due to addition of the solvent may be reduced.

For example, in the method of forming a thin film, the reduction rate of thin film growth rate per cycle (Å/Cycle) calculated by Equation 1 below is-5% or less, preferably-10% or less, more preferably-20% or less, still more preferably-30% or less, still more preferably-40% or less, most preferably-45% or less. Within this range, step coverage and the thickness uniformity of the film may be excellent.

Reduction rate of thin film growth rate per cycle (%)=[(Thin film growth rate per cycle when film quality improver is used−Thin film growth rate per cycle when film quality improver is not used)/Thin film growth rate per cycle when film quality improver is not used]×100 [Equation 1]

In Equation 1, the thin film growth rate per cycle when using and not using the film quality improver refers to the thin film deposition thickness per cycle (Å/cycle), that is, the deposition rate. For example, when measuring the deposition rate, the final thickness of the thin film may be measured by ellipsometry, and the average deposition rate may be obtained by dividing the measured value by the total number of cycles.

In Equation 1, “when the film quality improver is not used” refers to a case where a thin film is formed by adsorbing only the thin film precursor compound onto the substrate in the thin film deposition process. As a specific example, in the method of forming a thin film, “when the film quality improver is not used” refers to a case where a thin film is formed by omitting the step of adsorbing the film quality improver and the step of purging the non-adsorbed film quality improver.

In the method of forming a thin film, based on a thin film thickness of 100 Å measured by SIMS, residual halogen intensity (c/s) in a thin film may be preferably 100,000 or less, more preferably 70,000 or less, still more preferably 50,000 or less, still more preferably 10,000 or less, as a preferred example, 5,000 or less, more preferably 1,000 to 4,000, still more preferably 1,000 to 3,800. Within this range, corrosion and deterioration may be prevented.

In the present disclosure, purging may be performed preferably at 1,000 to 50,000 sccm (Standard Cubic Centimeter per Minute), more preferably 2,000 to 30,000 sccm, still more preferably 2,500 to 15,000 sccm. Within this range, thin film growth rate per cycle may be appropriately controlled. In addition, an atomic mono-layer or a similar type of layer may be formed through deposition, which is advantageous in terms of film quality.

The atomic layer deposition (ALD) process is very advantageous in fabricating integrated circuits (ICs) requiring a high aspect ratio, and in particular, due to a self-limiting thin film growth mechanism, excellent conformality and uniformity and precise thickness control may be achieved.

For example, in the method of forming a thin film, the deposition temperature may be 50 to 800° C., preferably 300 to 700° C., more preferably 400 to 650° C., still more preferably 400 to 600° C., still more preferably 450 to 600° C. Within this range, an effect of growing a thin film having excellent film quality may be obtained while implementing ALD process characteristics.

For example, in the method of forming a thin film, the deposition pressure may be 0.01 to 20 Torr, preferably 0.1 to 20 Torr, more preferably 0.1 to 10 Torr, most preferably 1 to 7 Torr. Within this range, a thin film having a uniform thickness may be obtained.

In the present disclosure, the deposition temperature and the deposition pressure may be temperature and pressure in a deposition chamber or temperature and pressure applied to a substrate in a deposition chamber.

The method of forming a thin film may preferably include a step of increasing temperature in a chamber to a deposition temperature before introducing the film quality improver into the chamber; and/or a step of performing purging by injecting an inert gas into the chamber before introducing the film quality improver into the chamber.

In addition, as a thin film-forming apparatus capable of implementing the method of forming a thin film, the present invention may include a thin film-forming apparatus including an ALD chamber, a first vaporizer for vaporizing a film quality improver, a first transfer means for transferring the vaporized film quality improver into the ALD chamber, a second vaporizer for vaporizing a thin film precursor, and a second transfer means for transferring the vaporized thin film precursor into the ALD chamber. Here, vaporizers and transfer means commonly used in the art to which the present invention pertains may be used without particular limitation.

As a specific example, the method of forming a thin film is described in detail as follows.

First, a substrate on which a thin film is to be formed is placed in a deposition chamber capable of performing atomic layer deposition.

The substrate may include a semiconductor substrate such as a silicon substrate or a silicon oxide substrate.

A conductive layer or an insulating layer may be further formed on the substrate.

To deposit a thin film on the substrate placed in the deposition chamber, the film quality improver and a thin film precursor compound or a mixture of the thin film precursor compound and a non-polar solvent are prepared, respectively.

Then, the prepared film quality improver is injected into a vaporizer, converted into a vapor phase, transferred to a deposition chamber, and adsorbed on the substrate. Then, the non-adsorbed film quality improver is purged.

Next, the prepared thin film precursor compound or a mixture (composition for forming a thin film) of the thin film precursor compound and a non-polar solvent is injected into a vaporizer, converted into a vapor phase, transferred to a deposition chamber, and adsorbed on the substrate. Then, the non-adsorbed thin film precursor compound/composition for forming a thin film is purged.

In the present disclosure, the process of adsorbing the film quality improver on the substrate and performing purging to remove the non-adsorbed film quality improver and the process of adsorbing the thin film precursor compound on the substrate and performing purging to remove the non-adsorbed thin film precursor compound may be performed by changing the order thereof as needed.

In the present disclosure, when the film quality improver and the thin film precursor compound (composition for forming a thin film) are transferred to a deposition chamber, a vapor flow control (VFC) method using a mass flow control (MFC) method, or a liquid delivery system (LDS) using a liquid mass flow control (LMFC) method may be used. Preferably, the LDS method is used.

In this case, one selected from argon (Ar), nitrogen (N₂), and helium (He) or a mixed gas of two or more thereof may be used as a transport gas or a diluent gas for moving the film quality improver or the thin film precursor compound onto the substrate, but the present invention is not limited thereto.

In the present disclosure, for example, an inert gas may be used as the purge gas, and the transport gas or the dilution gas may be preferably used as the purge gas.

Next, a reaction gas is supplied. Reaction gases commonly used in the art to which the present invention pertains may be used as the reaction gas of the present invention without particular limitation. Preferably, the reaction gas may include a reducing agent, a nitrifying agent, or an oxidizing agent. A metal thin film is formed by reacting the reducing agent with the thin film precursor compound adsorbed on the substrate, a metal nitride thin film is formed by the nitrifying agent, and a metal oxide thin film is formed by the oxidizing agent.

Preferably, the reducing agent may be an ammonia gas (NH₃) or a hydrogen gas (H₂), the nitrifying agent may be a nitrogen gas (N₂), a hydrazine gas (N₂H₄), or a mixture of a nitrogen gas and a hydrogen gas, and the oxidizing agent may include one or more selected from the group consisting of H₂O, H₂O₂, O₂, O₃, and N₂O.

Next, the unreacted residual reaction gas is purged using an inert gas. Accordingly, in addition to the excess reaction gas, produced by-products may also be removed.

As described above, in the method of forming a thin film, for example, the step of adsorbing a film quality improver on a substrate, the step of purging the non-adsorbed film quality improver, the step of adsorbing a thin film precursor compound/composition for forming a thin film on the substrate, the step of purging the non-adsorbed thin film precursor compound/composition for forming a thin film, the step of supplying a reaction gas, and the step of purging the remaining reaction gas may be set as a unit cycle. The unit cycle may be repeatedly performed to form a thin film having a desired thickness.

As another example, in the method of forming a thin film, the step of adsorbing a thin film precursor compound/composition for forming a thin film on a substrate, the step of purging the non-adsorbed thin film precursor compound/composition for forming a thin film, the step of adsorbing a film quality improver on the substrate, the step of purging the non-adsorbed film quality improver, the step of supplying a reaction gas, and the step of purging the remaining reaction gas may be set as a unit cycle. The unit cycle may be repeatedly performed to form a thin film having a desired thickness.

For example, the unit cycle may be performed 1 to 99,999 times, preferably 10 to 1,000 times, more preferably 50 to 5,000 times, still more preferably 100 to 2,000 times. Within this range, desired thin film properties may be effectively expressed.

In addition, the present invention provides a semiconductor substrate. The semiconductor substrate is fabricated using the method of forming a thin film of the present invention. In this case, the step coverage, thickness uniformity, density, and electrical properties of a thin film may be excellent.

Preferably, the formed thin film has a thickness of 30 nm or less, a resistivity value of 15 to 400 μΩ·cm based on a thin film thickness of 10 nm, a halogen content of 10,000 ppm or less, and a step coverage of 80% or more. Within this range, the thin film has excellent performance as a diffusion barrier and may reduce corrosion of metal wiring materials, but the present invention is not limited thereto.

For example, the thin film may have a thickness of 1 to 30 nm, preferably 2 to 27 nm, more preferably 3 to 25 nm, still more preferably 5 to 23 nm. Within this range, thin film properties may be excellent.

For example, based on a thin film thickness of 10 nm, the thin film may have a resistivity value of 10 to 400 μΩ·cm, preferably 15 to 300 μΩ·cm, more preferably 20 to 290 μΩ·cm, still more preferably 25 to 280 μΩ·cm. Within this range, thin film properties may be excellent.

The thin film may have a halogen content of preferably 1,000 ppm or less or 1 to 1,000 ppm, still more preferably 5 to 500 ppm, still more preferably 10 to 100 ppm. Within this range, thin film properties may be excellent, and corrosion of metal wiring materials may be reduced. Here, for example, the halogen remaining in the thin film may be Cl₂, Cl, or Cl⁻. As the amount of halogen remaining in the thin film decreases, film quality increases.

For example, the thin film may have a step coverage of 80% or more, preferably 90% or more, more preferably 92% or more. Within this range, even a thin film with a complicated structure may be easily deposited on a substrate, which has the advantage of being applicable to next-generation semiconductor devices.

For example, the formed thin film may include one or more selected from the group consisting of a titanium nitride film (TiₓN_{y}, 0<x≤1.2, 0<y≤1.2, preferably 0.8≤x≤1, 0.8≤y≤1, more preferably x and y are 1) and a titanium oxide film (TiO₂), preferably a titanium nitride film. In this case, the thin film may be usefully used as a diffusion barrier, an etch stop film, or an electrode for semiconductor devices.

For example, when necessary, the thin film may have a multilayer structure consisting of two or three layers. As a specific example, the multilayer structure consisting of two layers may be a lower layer-middle layer structure, and the multilayer structure consisting of three layers may be a lower layer-middle layer-upper layer structure.

For example, the lower layer may be composed of one or more selected from the group consisting of Si, SiO₂, MgO, Al₂O₃, Cao, ZrSiO₄, ZrO₂, HfSiO₄, Y₂O₃, HfO₂, LaLuO₂, Si₃N₄, SrO, La₂O₃, Ta₂O₅, BaO, and TiO₂.

For example, the middle layer may be composed of TiₓN_{y}, preferably TN.

For example, the upper layer may be composed of one or more selected from the group consisting of W and Mo.

Hereinafter, the present invention will be described in more detail with reference to the following preferred examples and drawings. However, these examples and drawings are provided for illustrative purposes only and should not be construed as limiting the scope and spirit of the present invention. In addition, it will be apparent to those skilled in the art that various changes and modifications may be made without departing from the spirit and scope of the present invention, and such changes and modifications are also within the scope of the appended claims.

### EXAMPLES

### Examples 1 and 2 and Comparative Examples 1 to 4

The compounds shown in Table 1 were prepared as film quality improvers, and TiCl₄ was prepared as a thin film precursor compound. The prepared film quality improver was placed in a canister and supplied to a vaporizer heated to 150° C. at a flow rate of 0.05 g/min using a liquid mass flow controller (LMFC) at room temperature. The film quality improver vaporized in the vaporizer was fed into a deposition chamber loaded with a substrate for 1 second, and then argon gas was supplied thereto at 5,000 sccm for 2 seconds to perform argon purging. At this time, the pressure in the reaction chamber was controlled to 2.5 torr. Next, the prepared TiCl₄ was placed in a separate canister and supplied to a separate vaporizer heated to 150° C. at a flow rate of 0.05 g/min using a liquid mass flow controller (LMFC) at room temperature. The TiCl₄ vaporized in the vaporizer was fed into the deposition chamber for 1 second, and then argon gas was supplied thereto at 5,000 sccm for 2 seconds to perform argon purging. At this time, the pressure in the reaction chamber was controlled to 2.5 torr. Next, after introducing ammonia as a reactive gas into the reaction chamber at 1,000 sccm for 3 seconds, argon purging was performed for 3 seconds. At this time, the substrate on which a metal thin film is to be formed was heated to 460° C. This process was repeated 200 to 400 times to form a 10 nm-thick TiN thin film as a self-limiting atomic layer.

However, in Comparative Example 1, the step of adsorbing the film quality improver and the step of performing purging to remove the non-adsorbed film quality improver after adsorbing the film quality improver were omitted.

TABLE 1

| Classification | Film quality improver (growth inhibitor) |
|---|---|
| Example 1 | 2-Chloro-2-methyl butane |
| Example 2 | 3-Chloro-3-methyl pentane |
| Comparative Example 1 | — |
| Comparative Example 2 | Tert-butyl chloride |
| Comparative Example 3 | 1,2,3-Trichloropropane |
| Comparative Example 4 | 2-Chloropropane |

### Test Examples

### 1) Deposition Evaluation (Average Deposition Rate and Decrease Rate in Thin Film Growth Rate)

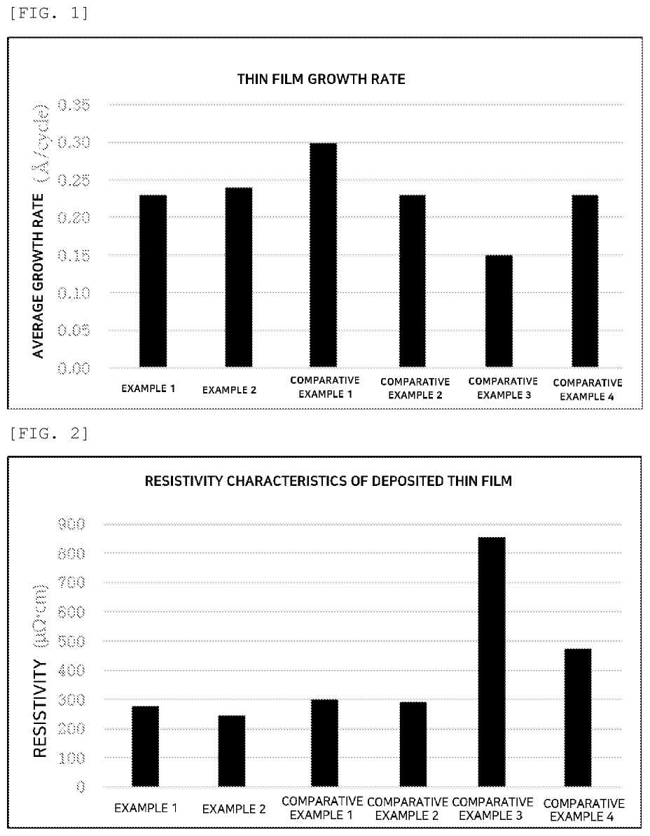

An ellipsometer capable of measuring the optical properties of a thin film including thickness and refractive index using the polarization characteristics of light was used to measure the thickness of the formed thin film. The thickness of the thin film deposited per cycle was calculated by dividing the measured thickness value by the number of cycles. Based on the calculated values, deposition rate was evaluated, and the results are shown in Table 2 and FIG. 1 below.

In addition, the reduction rate of thin film growth rate per cycle was calculated by substituting the measured deposition rate per cycle into Equation 1a below, and the results are shown in Table 2 below.

Reduction rate of thin film growth rate per cycle (%)=[(Thin film growth rate per cycle when film quality improver is used−Thin film growth rate per cycle of Comparative Example 1)/Thin film growth rate per cycle of Comparative Example 1]×100 [Equation 1a]

In Equation 1a, when using the film quality improver and when not using the film quality improver (Comparative Example 1), the thin film growth rate per cycle means the thin film deposition thickness per cycle (Å/cycle). The thin film deposition thickness per cycle is the average deposition rate value obtained by dividing the thin film thickness measured above by the total number of cycles.

### 2) Evaluation of Thin Film Resistance (Resistivity)

The surface resistance of the formed thin film was measured using the four-point probe method to obtain sheet resistance, and then the resistivity value was calculated from the thickness value of the thin film.

TABLE 2

| | | Deposi- | Reduction rate | |
|---|---|---|---|---|
| | | tion | of thin film | Resis- |
| Classifica- | Film quality | rate | growth rate per | tivity |
| tion | improver | (Å/cycle) | cycle (GPC) (%) | (μΩ · cm) |
| Example 1 | 2-Chloro-2- | 0.23 | −23 | 276 |
| | methyl butane | | | |
| Example 2 | 3-Chloro-3- | 0.24 | −20 | 244 |
| | methyl pentane | | | |
| Comparative | — | 0.30 | — | 300 |
| Example 1 | | | | |
| Comparative | Tert-butyl | 0.23 | −23 | 293 |
| Example 2 | chloride | | | |
| Comparative | 1,2,3- | 0.15 | −50 | 857 |
| Example 3 | Trichloropro- | | | |
| | pane | | | |
| Comparative | 2-Chloropro- | 0.23 | −23 | 474 |
| Example 4 | pane | | | |

As shown in Table 2 and FIGS. 1 and 2 below, in the case of Examples 1 and 2 using 2-chloro-2-methyl butane and 3-chloro-3-methyl pentane as the film quality improver of the present invention, compared to Comparative Example 1 not using the film quality improver, the deposition rate decreased by 20 to 23%, and the resistivity decreased to about 24 to 56 μΩ·cm. Based on these results, it was confirmed that the thin film growth appropriately controlled and the electrical properties were improved.

In contrast, in the case of Comparative Example 2 using a tert-butyl halogenated compound as the film quality improver, the deposition rate was reduced, and the resistivity exceeded 290 μΩ·cm. In the case of Comparative Example 3 using 1, 2, 3-trichloropropane as the film quality improver, the deposition rate was the smallest, and the resistivity value increased dramatically, indicating that the electrical properties of the thin film was not improved. In the case of Comparative Example 4, compared to Comparative Example 1, the deposition rate or the resistivity value was not reduced, indicating that the film quality was not improved.

### 3) Impurity Reduction Characteristics

SIMS analysis was performed on Cl and C elements to compare the impurities of the formed thin film, that is, process by-product reduction characteristics, and the results are shown in Table 3 below.

### 4) Density of Thin Film

The density of the formed thin film was measured based on X-ray reflectometry (XRR) analysis, and the results are shown in Table 3 below.

TABLE 3

| | | Intensity of impurities | | Density of |
|---|---|---|---|---|
| | Film quality | (Counts/s) | | thin film |
| Classification | improver | Cl | C | (g/mL) |
| Example 1 | 2-Chloro-2- | 3,413 | 392 | 5.06 |
| | methylbutane | | | |
| Example 2 | 3-Chloro-3-methyl | 3,170 | 395 | 5.18 |
| | pentane | | | |
| Comparative | — | 3,910 | 400 | 5.00 |
| Example 1 | | | | |
| Comparative | Tert-butyl chloride | 3,613 | 398 | 4.99 |
| Example 2 | | | | |
| Comparative | 1,2,3- | 5,000 | 23,500 | — |
| Example 3 | Trichloropropane | | | |
| Comparative | 2-Chloropropane | 5,000 | 500 | — |
| Example 4 | | | | |

* Standard thickness of sample thin film: 10 nm

As shown in Table 3, in the case of Examples 1 and 2 using the film quality improver according to the present invention, compared to Comparative Example 1 not using the film quality improver, the intensities of Cl and C decreased, indicating that impurity reduction characteristics were excellent. In particular, in the case of Comparative Example 1, since no carbon-containing compounds were added in the thin film deposition process, no carbon should have been detected in theory. However, carbon, presumed to originate from trace amounts of CO and/or CO₂ contained in the thin film precursor compound, purge gas, and reaction gas, was detected. In Examples 1 and 2 of the present invention, the carbon intensity decreased compared to Comparative Example 1 even though a film quality improver, which is a hydrocarbon compound, was added during thin film deposition. These results indicate that the film quality improver of the present invention has excellent impurity reduction characteristics.

In addition, the thin film density of Examples 1 and 2 was found to be higher than that of Comparative Example 1. These results show that the thin film density is increased by the film quality improver of the present invention. Accordingly, even when the thin film of the present invention is applied to a substrate requiring a high aspect ratio, excellent electrical properties may be obtained. In addition, when the thin film of the present invention is applied to a diffusion barrier or an etch stop film, barrier properties may be excellent.

On the other hand, in the case of Comparative Example 2, a compound with a structure similar to that of the film quality improver of the present invention was added, but the intensity of impurities increased compared to Examples 1 and 2, and there was no effect of improving thin film density. In the case of Comparative Example 3 and 4, compared to Comparative Example 1, it was confirmed that the intensity of impurities was too high and there was no effect of improving film quality.

1. A film quality improver, wherein the film quality improver is a compound represented by Chemical Formula 1 below: 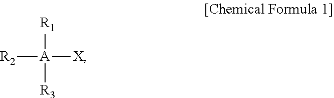
wherein A is carbon or silicon, R₁, R₂, and R₃ are independently alkyl groups having 1 to 3 carbon atoms, one or more of R₁, R₂, and R₃ has 2 or 3 carbon atoms, and X comprises one or more of fluorine (F), chlorine (CI), bromine (Br), and iodine (I).

2. The film quality improver according to claim 1, wherein X is fluorine, chlorine, or bromine.

3. The film quality improver according to claim 1, wherein X is iodine.

4. The film quality improver according to claim 1, wherein any one of R₁, R₂, and R₃ has 1 carbon atom, and the remaining two have 2 or 3 carbon atoms.

5. The film quality improver according claim 1, wherein the compound represented by Chemical Formula 1 is used in an atomic layer deposition (ALD) process.

6. A method of forming a thin film, comprising injecting a film quality improver represented by Chemical Formula 1 below into an ALD chamber and adsorbing the film quality improver on a surface of a substrate loaded into the ALD chamber: 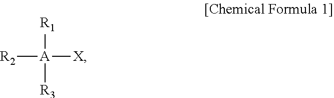
wherein A is carbon or silicon, R₁, R₂, and R₃ are independently alkyl groups having 1 to 3 carbon atoms, one or more of R₁, R₂, and R₃ has 2 or 3 carbon atoms, and X comprises one or more of fluorine (F), chlorine (Cl), bromine (Br), and iodine (I).

7. The method according to claim 6, comprising:
step i) of vaporizing the film quality improver and adsorbing the film quality improver on a surface of a substrate loaded into an ALD chamber;
step ii) of performing first purging of an inside of the ALD chamber using a purge gas;
step iii) of vaporizing a thin film precursor compound and adsorbing the thin film precursor compound on the surface of the substrate loaded into the ALD chamber;
step iv) of performing second purging of the inside of the ALD chamber using a purge gas;
step v) of supplying a reaction gas into the ALD chamber; and
step vi) of performing third purging of the inside of the ALD chamber using a purge gas.

8. The method according to claim 6, comprising:
step i) of vaporizing a thin film precursor compound and adsorbing the thin film precursor compound on a surface of a substrate loaded into an ALD chamber;
step ii) of performing first purging of an inside of the ALD chamber using a purge gas;
step iii) of vaporizing the film quality improver and adsorbing the film quality improver on the surface of the substrate loaded into the ALD chamber;
step iv) of performing second purging of the inside of the ALD chamber using a purge gas;
step v) of supplying a reaction gas into the ALD chamber; and
step vi) of performing third purging of the inside of the ALD chamber using a purge gas.

9. The method according to claim 7, wherein an amount of the purge gas introduced into the ALD chamber in step ii) is 10 to 100,000 times a volume of the film quality improver introduced in step i).

10. The method according to claim 8, wherein an amount of the purge gas introduced into the ALD chamber in step iv) is 10 to 100,000 times a volume of the film quality improver introduced in step iii).

11. The method according to claim 7, wherein the film quality improver and the thin film precursor compound are transferred into the ALD chamber by a VFC method, a DLI method, or an LDS method.

12. The method according to claim 7, wherein, when the film quality improver and the precursor compound are introduced into the ALD chamber, an input amount ratio (mg/cycle) of the film quality improver to the precursor compound is 1:1.5 to 1:20.

13. The method according to claim 7, wherein a reduction rate of thin film growth rate per cycle (Å/Cycle) calculated by Equation 1 below is −5% or less:
Reduction rate of thin film growth rate per cycle (%)=[(Thin film growth rate per cycle when film quality improver is used−Thin film growth rate per cycle when film quality improver is not used)/Thin film growth rate per cycle when film quality improver is not used]×100 [Equation 1]

14. The method according to claim 7, wherein an intensity (c/s) of halogen remaining in a thin film formed after 200 cycles measured according to SIMS is 10,000 or less.

15. The method according to claim 7, wherein the reaction gas is a reducing agent, a nitriding agent, or an oxidizing agent.

16. A semiconductor substrate fabricated using the method according to claim 6.

17. The semiconductor substrate according to claim 16, wherein the thin film has a thickness of 30 nm or less, a resistivity value of 10 to 400 μΩ·cm based on a thin film thickness of 10 nm, a halogen content of 1,000 ppm or less, and a step coverage of 80% or more.

18. The semiconductor substrate according to claim 16, wherein the thin film comprises a titanium nitride film.

19. The semiconductor substrate according to claim 16, wherein the thin film has a multilayer structure consisting of two or three layers.
